(19) United States
(12) Patent Application Publication
(10) Pub. No.: US 2021/0244955 A1
(43) Pub. Date: Aug. 12, 2021

(54) Primates Herpes Zoster Treatment Apparatus and Driving Method Thereof

(71) Applicant: Whi Young KIM, Busan (KR)

(72) Inventor: Whi Young KIM, Busan (KR)

(21) Appl. No.: 16/802,592

(22) Filed: Feb. 27, 2020

(30) Foreign Application Priority Data
Feb. 7, 2020 (KR) 10-2020-0014984

(51) Int. Cl. A61N 2/00; A61N 2/02; A61D 99/00

(52) U.S. Cl. CPC A61N 2/004; A61D 99/00; A61N 2/008; A61N 2/02

(57) ABSTRACT

A primates herpes zoster treatment apparatus according to an embodiment of the present invention includes: a multiplication voltage generator multiplying a designated voltage into voltages with different high frequencies; a first treatment stimulation coil unit treating herpes zoster by outputting a magnetic stimulation pulse by the multiplied voltage with a first frequency; a second treatment stimulation coil unit treating a herpes zoster post-pain by outputting a magnetic stimulation pulse by the multiplied voltage with a second frequency; and a controller selectively driving the first treatment stimulation coil unit and the second treatment stimulation coil unit.

### BACKGROUND OF THE INVENTION

### Field of the Invention

The present invention relates to a primates herpes zoster treatment apparatus and a driving method thereof and, more particularly, to a primates herpes zoster treatment apparatus that simultaneously treats herpes zoster and herpes zoster post-pain and a driving method thereof.

### Description of the Related Art

Nerve damage or inflammation of a tissue is chemically and anatomically coupled and the sympathetic nerve system is involved in pain due to coupling of an efferent sympathetic nerve fiber and a primary afferent nerve cell during an abnormal activation or reproduction process of an alpha adrenaline receptor of the primary afferent nerve cell. Acute phase herpes zoster treatment treats a skin lesion, adjusts pain, and prevents progression to neuralgia after herpes zoster, and prevents progression to neuralgia after herpes zoster by adjusting an acute phase pain and preventing central sensitization, so pain adjustment has an important role.

Treatment methods of the related art give an antiviral drug in the early stage, but it is effect within 50 hours and steroid is orally administered with the antiviral drug to prevent progression of inflammation reaction and nerve damage. However, it has been known that when they are simultaneously administered, there is an effect of reducing an acute pain, but it cannot prevent generation of post nerve damage of herpes zoster.

According to nerve block, the sympathetic nerve system is not involved in pain and sympathetic nerve stimulation is generated and the amount of blood flowing to the nerve cells is reduced due to an acute inflammation reaction. Further, herpes zoster is naturally cured in many cases, and unclear nerve block and a continuous pain cause sensitization of the nervous system, which causes progression to chronic pain, so it is important to suppress pain as soon as possible.

### Documents of Related Art

(Patent Document 0001) Korean Patent Application Publication No. 10-2009-0125748 (2009 Dec. 7)

(Patent Document 0002) Korean Utility Model Registration No. 20-0374923 (2005 Jan. 26)

### SUMMARY OF THE INVENTION

An object of the present invention is to provide a primates herpes zoster treatment apparatus that simultaneously treats herpes zoster and herpes zoster post-pain, and driving method thereof.

A primates herpes zoster treatment apparatus according to an embodiment of the present invention includes: a multiplication voltage generator multiplying a designated voltage into voltages with different high frequencies; a first treatment stimulation coil unit treating herpes zoster by outputting a magnetic stimulation pulse by the multiplied voltage with a first frequency; a second treatment stimulation coil unit treating a herpes zoster post-pain by outputting a magnetic stimulation pulse by the multiplied voltage with a second frequency; and a controller selectively driving the first treatment stimulation coil unit and the second treatment stimulation coil unit.

The first treatment stimulation coil unit and the second treatment stimulation coil unit may be operated with a switching unit that is controlled to perform switching by the controller.

The multiplication voltage generator may include a high-voltage multiplier multiplying voltage into high voltages with different frequency and then providing the voltages to the switching unit.

The multiplication voltage generator may include: a start current compensator compensating for a start current by the designated voltage; a transformer generating a high voltage from the designated voltage; and a ZCS resonance inverter connected to a primary coil of the transformer and the start current compensator and performing a resonance operation by ZCS (zero-current switching).

When the ZCS resonance inverter is operated, voltage induced by a secondary coil of the transformer may be input to the high-voltage multiplier.

The multiplication voltage generator may use a full-wave Cockcroft Walton circuit as the high-voltage multiplier.

A method of driving a primates herpes zoster treatment apparatus according to an embodiment of the present invention includes: multiplying a designated voltage into voltages with different frequencies by means of a multiplication voltage generator; treating herpes zoster by outputting a magnetic stimulation pulse by the multiplied high voltage with a first frequency by means of a first treatment stimulation coil unit; treating a herpes zoster post-pain by outputting a magnetic stimulation pulse by the multiplied high voltage with a second frequency by means of a second treatment stimulation coil unit; and selectively driving the first treatment stimulation coil unit and the second treatment stimulation coil unit by means of a controller.

The method may further include operating the first treatment stimulation coil unit and the second treatment stimulation coil unit with a switching unit that is controlled to perform switching by the controller.

The multiplication voltage generator may use a high-voltage multiplier multiplying voltage into high voltages with different frequency and then providing the voltages to the switching unit.

The multiplying may include: compensating for a start current by the designated voltage by means of a start current compensator; generating a high voltage from the designated voltage by means of a transformer; and performing a resonance operation by ZCS by means of a ZCS resonance inverter connected to a primary coil of the transformer and the start current compensator.

When the ZCS resonance inverter is operated, voltage induced by a secondary coil of the transformer may be input to the high-voltage multiplier.

The multiplication voltage generator may use a full-wave Cockcroft Walton circuit as the high-voltage multiplier.

In an embodiment of the present invention, as the result of trying to implement and connecting a concept in consideration of the fact that active treatment access is required to recover motor disturbance related to herpes zoster and manage a herpes zoster post-pain in an early stage, by applying a magnetic field to a body, it is possible to treat pain by generating eddy currents at a level that can stimulate muscles and nerve cells, so the present invention is effective to incurable diseases and can be widely used for various pain treatments.

Further, according to an embodiment of the present invention, there is an effect of improving blood flow, necrosis of Human Leukemic cell occurs in a magnetic field, and realizes promotion of cure and reduction of pain. Further, movement of ions, etc. through cell membranes easily increases activity of phagocytes and enzymes, induces depolarization of cell membranes, changes membrane potential, secretes growth factors, transmits calcium ions, and synthesis of chondrocytes, and enables pain treatment.

Ion activation not only improves blood flow, but treats diseases and removes the reasons of pain by permeating into deep parts and stimulating nerves, muscles, and skeletal tissues, and converts acid body fluid into alkali body fluid. Capillaries not only pass blood, but also operates from a closed state, blood is circulated by arteriovenous anastomosis and selectively flows through capillaries due to metabolism required by tissues, the role there of is determined in accordance with the degrees of contraction and stretch of arterioles or capillaries, and the degrees of contraction and stretch are adjusted by the metabolism of blood vessels in tissues and the sympathetic nerves of the smooth muscle of blood vessels. Accordingly, contraction and stretch of arterioles and capillaries are directly generated by stimulating sympathetic nerves and a sympathetic signal transmitted to the brain through the spinal cord influences contraction of blood vessels of skin sympathetic nerves and activity of expansion fibers, thereby being able to increase the amount of blood in the capillaries and the blood vessels.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a diagram showing the entire configuration of a primates herpes zoster treatment apparatus according to an embodiment of the present invention;

FIG. 2 is a diagram showing the options of an assistant image processing function;

FIG. 3 is a diagram showing the key concepts according to an embodiment of the present invention;

FIGS. 4A to 4D are diagram showing a driving process of the primates herpes zoster treatment apparatus of FIG. 1;

FIGS. 5 to 7 are diagrams showing a process for treatment, communication, control, and connection with an associated person; and

FIG. 8 is a flowchart showing a driving process of a primates herpes zoster treatment apparatus according to an embodiment of the present invention.

### DETAILED DESCRIPTION OF THE INVENTION

Hereinafter, embodiments of the present invention will be described in detail with reference to the accompanying drawings.

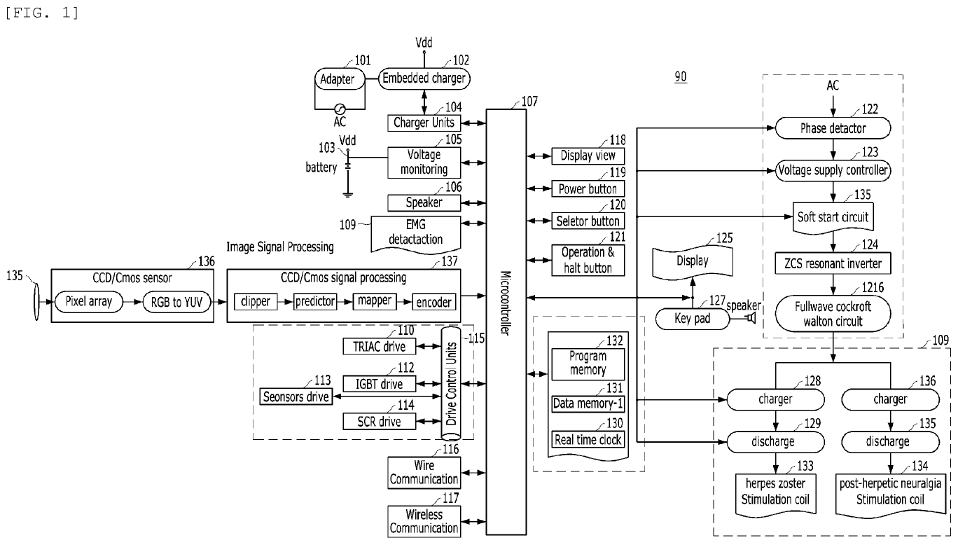

FIG. 1 is a diagram showing the entire configuration of a primates herpes zoster treatment apparatus according to an embodiment of the present invention.

As shown in FIG. 1, a primates herpes zoster treatment apparatus 90 according to an embodiment of the present invention includes some or all of a voltage part 101˜105, a sound output unit 106, a controller 107, a sensor (or sensing) unit 108, a sensor driver (or sensor module) 110, 112˜115, a communication unit 116, 117, a user interface 118˜121, multiplication voltage generator (or high-voltage generator) 122˜124, 126, 135, a memory 130˜132, a treatment stimulation coil unit 128, 129, 133, 134˜136, and an image signal processor 135˜137.

The term ‘including some or all’ means that the primates herpes zoster treatment apparatus 90 is configured without some components such as the memory 130˜132 or some components such as the sensor driver (or sensor module) 110, 112˜115 are included in other components such as the controller 107, and it is exemplified in the following description that all of them are included to help sufficiently understand the present invention.

The voltage part 101˜105 generates a voltage to be used for various components of the primates herpes zoster treatment apparatus 90. To this end, the voltage part 101˜105 includes some or all of an adapter 101, a built-in charger 102, a power battery 103 that is used in a mobile mode, a charge port 104 controlled by a microprocessor, and a voltage monitoring unit 105 performing a voltage monitoring function of the microprocessor.

The sound output unit 106 includes a speaker that outputs sound, and can output various items of voice information related to the operation of the primates herpes zoster treatment apparatus 90.

The controller 107 controls the general operations of various components of the primates herpes zoster treatment apparatus 90 and includes a microprocessor that is an important component. The controller 107 may include a CPU, an MPU, etc.

The sensing unit 108 may include an electromyogram (EMG) sensor for measuring EMG and a sensor module processing sensing data and can provide data in an option type to the microprocessor from the EMG sensor.

The sensor driver 110, 112˜115 includes some or all of a triac driver 110, an IGBT 112, a sensor driver 113 for driving various sensors, an SCR driver 114, and a driving control unit 115.

The communication unit 116, 117 may include a first communication unit 116 and a second communication unit 117. The first communication unit 116 is a wire communication port and the second communication unit 117 can perform a wireless communication port function.

The user interface 118˜121 includes a display view (or display) 118, a power button 119, a sensor button 120, and a start/stop button 121. The user interface 118˜121 can receive various control instructions for controlling the primates herpes zoster treatment apparatus 90. In addition, the user interface 118˜121 may further include a display window 125 that can be seen from the outside of a cranial nerve stimulation apparatus, that is, the primates herpes zoster treatment apparatus 90, and a keypad 127 that can be input through the display window 125. The display view 118 and the display window 125 may mean touch panels.

The multiplication voltage generator 122˜124, 126, 135 includes some or all of a phase detector 122 that detects a phase coming from AC, a voltage adjustment controller 123, a zero-current switching (ZCS) resonant converter 124, and a full-wave Cockcroft Walton circuit 126.

The full-wave Cockcroft Walton circuit 126 is a voltage multiplier (or frequency multiplier). The voltage multiplier is operated to generate high voltage and the operation principle is similar to that of a charge pump circuit. The full-wave Cockcroft Walton circuit 126 can output low-frequency voltage for treating herpes zoster and high-frequency voltage for treating herpes zoster post-pain. The low frequency and the high frequency may be considered as relative concepts, but when a reference frequency is used, a frequency lower than the frequency can be defined as a low frequency and a frequency higher than the reference frequency can be defined as a high frequency. The term ‘relative’ means that if a multiplier outputs high voltage with two different frequencies, the higher one becomes a high frequency. The concept of the term is not specifically limited in embodiments of the present invention.

The memory 130˜132 includes a real-time clock generator 130, a data memory region (unit) 131, and a program memory (unit) 132 driving the primates herpes zoster treatment apparatus 90. A real-time clock may be used, for example, to show the start of data processing and the program memory 132 can make a predetermined charging/discharging operation for providing a stimulation pulse according to an embodiment of the present invention be performed by executing internal programs.

The treatment stimulation coil unit 128, 129, 133, 134˜136 may include a first treatment stimulation coil unit and a second treatment stimulation coil unit for outputting low-frequency and high-frequency magnetic pulses, and may include a power semiconductor, etc. Reference numeral ‘128’ indicates storing the voltage from reference numeral ‘126’, reference numeral ‘129’ indicates discharging a power conductor, and reference numeral ‘133’ indicates a treatment stimulation coil unit for treating herpes zoster.

The treatment stimulation coil unit 128, 129, 133, 134˜136 includes a charger 136 that stores a rectified voltage, a discharger 135 that discharges the stored voltage, and a post-herpes zoster treatment stimulation unit coil unit 134. The treatment stimulation coil unit for treating herpes zoster can use low-frequency voltage and the post-herpes zoster treatment stimulation unit coil unit 134 can use high-frequency voltage.

In summary, herpes zoster is a disease caused by reactivation of varicella zoster virus (VZV), and shows propagation, movement revelation, and a vesicular lesion of a neuromuscular band shape in accordance with a sensory nerve. Infection incubates in sensory neurons and causes itching sensation, burning sensation, etc. along ganglia at the portions where the spine nerve, the fifth cranial nerve, and the seventh cranial nerve are distributed. When observing fluorescence having bioinformation of the skin that is absorbed in the skin through a real-time color image by detecting it with a high-sensitive CCD camera and radiating the blue light (400 nm) wavelength of visible light (400˜700 nm), since collagen, flavin, elastin, etc. that are components of the skin also contain fluorescent components, it is possible to measure, diagnose, and evaluate, using them, photo-aging, pigmentation, P-Acne, skin evenness, a skin disease, herpes zoster, and aging and it is also possible to easily determine pigmentation due to melanin. When herpes zoster and post-herpes zoster neuralgia appear, a severe pain and various motor disturbance are caused and patients with allodynia increase in the acute phase. When the period of nerve block increase after herpes zoster appears, there is a high possibility of appearance of post-herpes zoster neuralgia, a burning pain, an intermittent shooting pain, a lancinating pain, allodynia, etc. accompany, and chronic inflammation cells are shown in the dorsal root ganglion (DRG) and the peripheral nerves having the post-herpes zoster neuralgia.

Accordingly, in an embodiment of the present invention, the blood flow is improved through ion activation in the muscles, nerve cells, and capillaries using magnetic pulses, the acid body fluid is changed into an alkali body fluid, the arterioles and capillaries are contracted and stretched by simulating the sympathetic nerves, and a sympathetic signal transmitted to the brain through the spine influences the activity of contraction and expansion fibers of the sympathetic nerve blood vessels, thereby being able to increase the amount of blood in the capillaries and the blood vessels. This method reduces cell necrosis and movement of ions, etc. through cell membranes easily increases activity of phagocytes and enzymes, induces depolarization of cell membranes, changes membrane potential, secretes growth factors, transmits calcium ions, and synthesis of chondrocytes, and enables pain treatment. Further, a one-loop, that is, first treatment coil and a second treatment coil can be considered as providing an effective treatment method for herpes zoster and pain treatment with a lesion of post-herpetic neuralgia (PHN).

According to an embodiment of the present invention, a capacitor storing energy for stimulation and a coil generating an intensive magnetic field are provided. When a short and intensive current is applied to a stimulation coil, a pulse type magnetic field is formed and a time-varying magnetic field induces stimulation strength in the principle of stimulating nerves by inducing eddy current in the tissues in a human body, thereby generating a magnetic field of several tesla with pulses having hundreds of μs. Further, a treatment stimulation coil can be attached/detached in a cranial nerve stimulation apparatus, it is possible to control an EMG sensor equipped with an LE and a pulse device for stimulating a treatment pulse for pain treatment according to the skin and the lesion, and it is possible to add or replace a magnetic coil, depending on the performance and capacity of a power device.

Further, an EMG sensor module is a herpes zoster and pain treatment device that is optionally composed of an energy harvester and an EMG front end (analog), is configured in a small size for convenient carrying and moving, can immediately handle a disease when the symptoms are severe through monitoring according to the body conditions and connection with a hospital in cooperation with a cell phone.

In summary, after varicella is completely cured, it reappears after incubating in the dorsal root ganglion and the cranial ganglia, and the latent viruses are active sometimes, but are suppressed by cell mediated immunity. When cellular immunity decreases, the virus propagate in the nerve ganglia, destroy neurons, and induce pain while causing eruption and blisters. Accordingly, when a high-sensitive color camera is optionally provided for diagnosis, and when observing fluorescence having bioinformation of the skin and radiating the blue light (400 nm) wavelength of visible light (400˜700 nm), light is absorbed in porphyrin in herpes zoster, P. acne psoriasis, etc. and fluorescence is generated, and a method of applying a magnetic stimulation pulse to a lesion through detection, and recording and analyzing of images can be employed.

According to the primates herpes zoster treatment apparatus 90 of an embodiment of the present invention, when a magnetic pulse is applied to a human body, the blood flow is improved through ion activation in the muscles, nerve cells, and capillaries, the acid body fluid is changed into an alkali body fluid, the arterioles and capillaries are contracted and stretched by simulating the sympathetic nerves, and a sympathetic signal transmitted to the brain through the spine influences the activity of contraction and expansion fibers of the sympathetic nerve blood vessels, thereby being able to increase the amount of blood in the capillaries and the blood vessels.

This can reduce cell necrosis, movement of ions, etc. through cell membranes easily increases activity of phagocytes and enzymes, induces depolarization of cell membranes, changes membrane potential, secretes growth factors, transmits calcium ions, and synthesis of chondrocytes, and enables pain treatment. Further, a herpes zoster (e.g., one-loop, first loop) treatment coil and a post-zoster pain (e.g., two-loop, second loop) treatment coil can provide an effective treatment method for herpes zoster and pain treatment with a lesion of post-herpetic neuralgia (PHN).

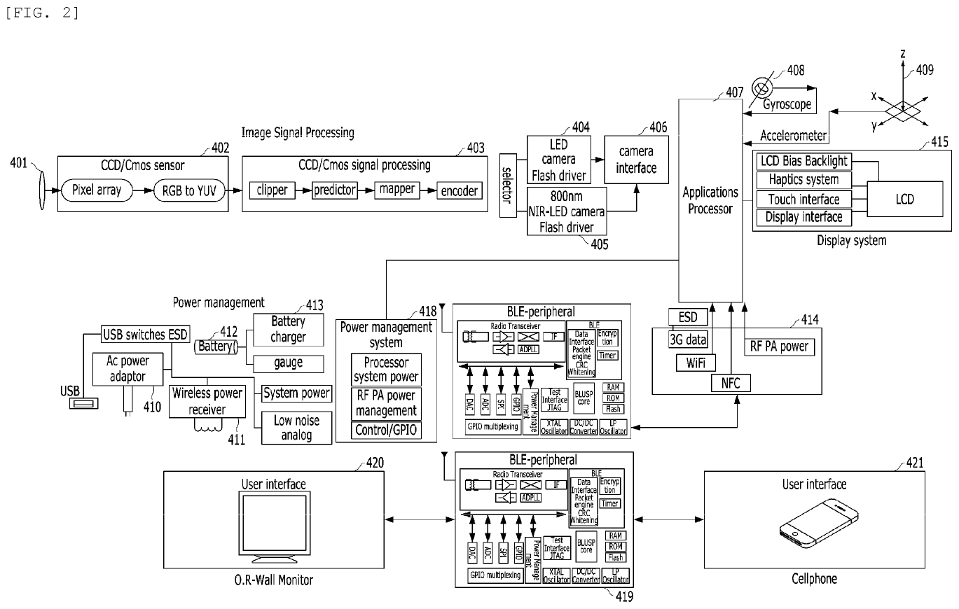

FIG. 2 is a diagram showing the options of an assistant image processing function and the function shown in FIG. 2 can be added to the function of the primates herpes zoster treatment apparatus 90 shown in FIG. 1.

Reference numeral ‘201’ denotes a lens, reference numeral ‘202’ denotes a CCD-CMOS sensor, reference numeral ‘203’ denotes a CCD-CMOS sensor signal processor, reference numeral ‘204’ denotes a common illumination LED, reference numeral ‘205’ denotes a near infrared ray LED driver of 700-1, 100 nm, reference numeral ‘206’ denotes a camera interface, reference numeral ‘207’ denotes an application processor, reference numeral ‘208’ denotes a gyroscope sensor for position correction, reference numeral ‘209’ denotes an accelerometer controlling the speed of position correction and posture correction, reference numeral ‘210’ denotes an AC adapter, reference numeral ‘211’ denotes a wireless power receiver, reference numeral ‘212’ denotes a the battery for wireless arthroscopy, reference numeral ‘213’ denotes a battery charger, reference numeral ‘214’ denotes a Bluetooth transmitter for transmitting/receiving small data of an auxiliary concept, reference numeral ‘215’ denotes an LCD control circuit, reference numeral ‘218’ denotes a power management system, reference numeral ‘219’ denotes a Bluetooth receiver for transmitting/receiving small data of an auxiliary concept, reference numeral ‘220’ denotes a monitor, and reference numeral ‘221’ denotes a cell phone.

An ultra wideband (UWB) communication technology is used as the image data transmission method that is the most suitable for transmitting high-quality images sensed in accordance with an embodiment of the present invention, and this is because the UWB communication in which a high quality has an ultra-wideband frequency band that can transmit a large amount of data at a time when data to be transmitted is large. Further, it is possible to diagnose and determine herpes zoster from the surface of a skin by simply applying the function of a camera.

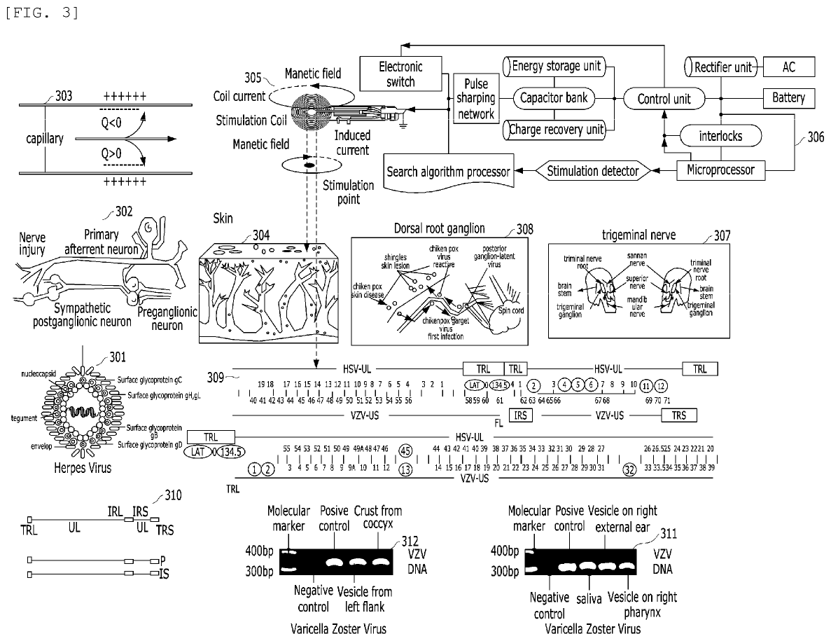

FIG. 3 is a diagram showing the key concepts according to an embodiment of the present invention.

Referring to FIG. 3, as key concepts proposed in an embodiment of the present invention, reference numeral ‘301’ denotes a herpes virus, reference numeral ‘302’ denotes a disease due to varicella zoster viruses of herpes zoster, and reference numeral ‘304’ denotes the dorsal root ganglia that manages dermatome of a lesion, which shows an acute skin disease in which an acute pain and bullous eruption appear along the sensory ganglia due to reactivation of viruses by factors such as deterioration of immunity, a physical damage, a malignant tumor.

Referring to reference numeral ‘303’, when a treatment pulse is applied to a human body, the blood flow is improved through ion activation in the muscles, nerve cells, and capillaries, the acid body fluid is changed into an alkali body fluid, the arterioles and capillaries are contracted and stretched by simulating the sympathetic nerves, and a sympathetic signal transmitted to the brain through the spine influences the activity of contraction and expansion fibers of the sympathetic nerve blood vessels, thereby being able to increase the amount of blood in the capillaries and the blood vessels.

Reference numeral ‘305’ denotes a treatment coil, reference numeral ‘306’ shows the function of a treatment pulse apparatus, reference numeral ‘304’ shows attenuation of pain and stop of appearance of herpes zoster viruses by discharge to a stimulation coil, reference numeral ‘307’ shows third nerve and spine ganglia, a peripheral sensory nerve, a dorsal root, acute inflammation of the sense of smell, cell permeation, swelling, inflammation of bleeding and ganglia necrosis in a severe case, and inflammation and degenerative degeneration, reference numeral ‘308’ shows a herpes virus that is latent in the dorsal root ganglia and causes an acute skin disease that causes an acute pain and bullous eruption along the sensory ganglia due to reactivation by factors. The herpes zoster virus (VZV) is an alpha herpes virus in the same lower family as herpes simplex viruses (HSV) 1, 2 of reference numeral ‘309, and the genome applies stimulation to a virus composed of an inherent long (UL) region and an inherent short (US) region limited by repetition of internal short (IRS) and short (TRS) of reference numeral ‘309’ divided into terminal long (TRL) and internal long (IRL) repeats.

The genome is linear in a virion with a non-pair nucleus at each end, an end pair and the genome circulate in a VZV infection cell, the genome has five repeated regions, in which a repeat region 1 (R1) is an open leading frame (ORF) 11 and R2 stimulates ORF14 (glycoprotein C), R3 of OFR22, R4 between ORF62 and the origin of virus reproduction, and R5 between ORF 60 and 61.

In reference number ‘310’, there is a peculiar long (UL) and inherent short area in which there is a repeat of terminal long (TRL), terminal short (TRS), internal long (IRL), and internal short (IRS), a VZV gene (second line) and HSV gene (third line) are indicated by numbers, a VZV without a humolog or the HSV gene of reference numeral ‘307’ is indicated by a circle, and an HSV genome includes UL and US areas and a repeat of TRL, TRS, IRL, and IRS.

Blisters actually appeared at the left flank and the tailbone of reference numeral ‘312’ in a patient with herpes zoster by a herpes zoster and post-zoster treatment apparatus for a VZV and an HSV, VZV DNA appeared in both of two lesions, and reference numeral ‘311’ shows the result of VZV DNA test from the blisters and the saliva of the patient, in which the blister appeared at the portion behind the right ear, the pharynx, and the occiput.

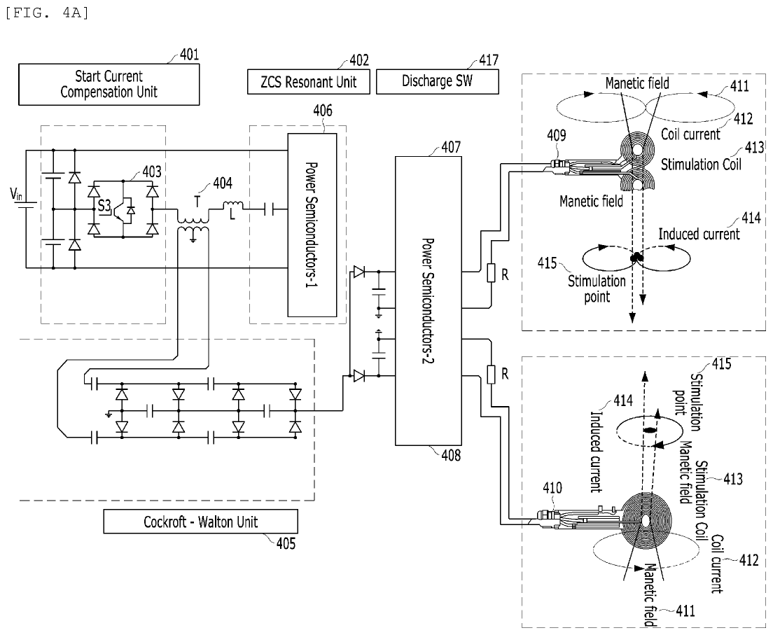

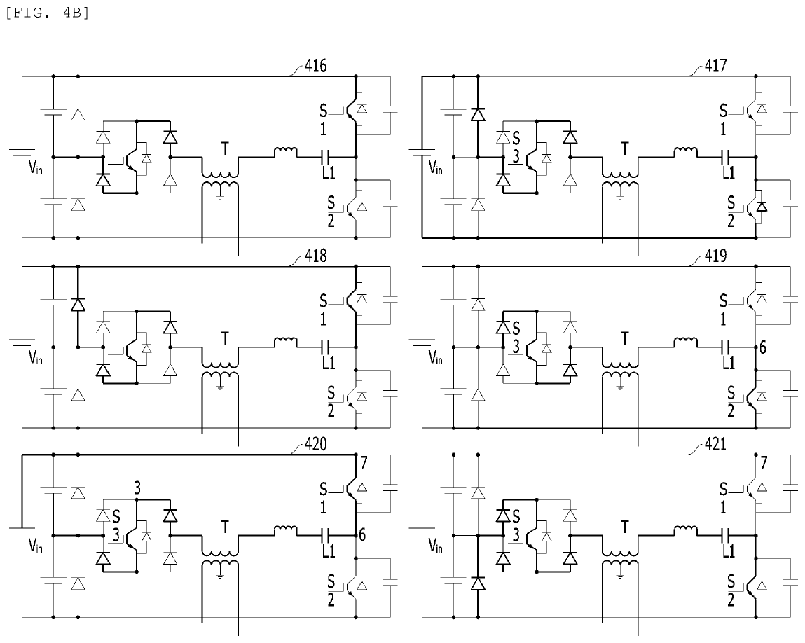

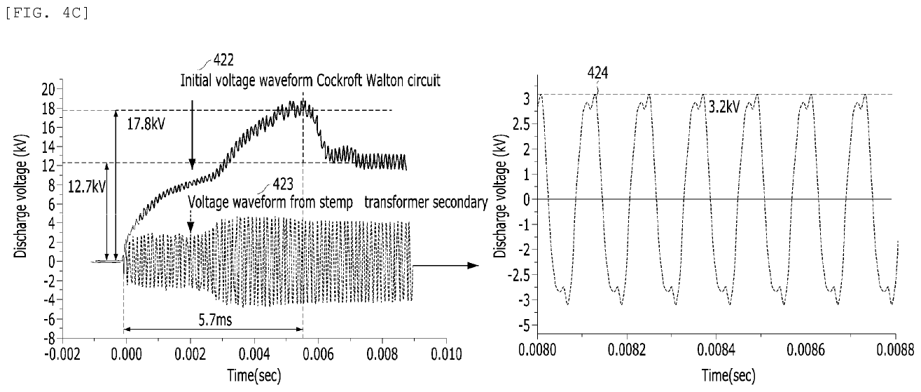

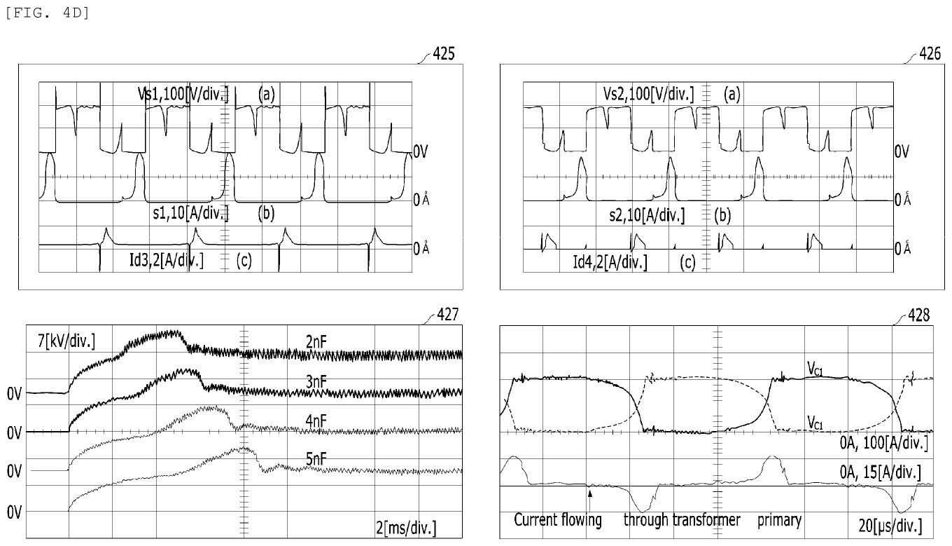

FIGS. 4A to 4D are diagram showing a driving process of the primates herpes zoster treatment apparatus of FIG. 1.

FIGS. 4A to 4D show a study and a test performed before a proposal according to an embodiment of the present invention. Reference numeral ‘401’ proposes a power circuit of a PWM inverter to suppress harmonic components output from a single-phase inverter under a DC power voltage and switching frequency condition by employing a starting current compensation circuit type and reference numeral ‘402’ shows proposes an inverter power circuit including a new starting power type of reference numeral ‘403’ in which output voltage shows not only zero and positive DC power voltage and negative DC power voltage and a half of positive DC voltage and a half of negative DC power voltage by appropriately changing a half bridge type power circuit. This method controls output voltage in a predetermined current mode by changing a control mode when a current over a rated current is required, an increasing frequency under the rated current can decrease voltage and reduce ripple by driving a multiplier stack for a switching power device under a load, an full-wave voltage doubler is implemented to reduce the ripple, and voltage drop and ripple factors are cut by effectively doubling the cycle per second and the number of charging. In particular, in reference numeral ‘402’, a current resonance type of half bridge inverter and a Cockcroft Walton circuit is employed into a high-voltage DC-DC converter, whereby a two-step voltage multiplier driven by a half bridge series resonance converter is obtained and a loss of switching is reduced. Further, since the number of winding turns of a step-up secondary transformer is small, the size is small and the parasitic capacity is small in a transformer stage, which is efficient.

In summary, reference numeral ‘403’ denotes a start current compensation circuit (unit) and reference numeral ‘404’ denotes a high-voltage step-up transformer, and reference numeral ‘4-5’ denotes a high-voltage multiplier using a Cockcroft Walton circuit. A ZCS series resonance inverter has a reduced loss due to tailing current generated when an IGBT is pulled and is composed of two IGBTs, a leakage inverter, a blocking capacitor, and a charging capacitor, the switching loss is fundamentally 0, a current through S1, S2, C1, and C2 is forced into a sine wave, a switch device is suitable for an operation with a high repetition speed because it is turned on or off at a zero-current, reference numeral ‘406’ functions as a blocking diode, reference numeral ‘407’ denotes an SCR that is a power device for high-frequency discharging, and reference numeral ‘408’ denotes an SCR that is a power device for low-frequency discharging. Reference numeral ‘409’ indicates a treatment coil of a 2-loop type primates herpes zoster and pain treatment apparatus, reference numeral ‘410’ indicates a treatment coil of a 1-loop type primates herpes zoster and pain treatment apparatus, reference numeral ‘411’ indicates a magnetic field, reference numeral ‘412’ indicates a coil current, reference numeral ‘413’ indicates a stimulation coil, reference numeral ‘414’ indicates an induces current, and reference numeral ‘415’ indicates a stimulation point. Reference numerals 416˜421 indicate an internal circuit operation. Reference numeral ‘422’ indicates a current flowing through a primary side of a transformer and a voltage of a charging capacitor, in which the current waveform is similar to a sine wave and shows a ZCS operation, a switching loss is considerably attenuated by the ZCS operation, a system start operation at an operation frequency of 8.3 kHz and an input voltage of 230V is shown, a discharging voltage is about 17.8 kV, this voltage is about 12.7 kV between electrodes of the apparatus, and a period of about 5.7 ms or 47.5 reaches a firing voltage. Reference numeral ‘424’ indicates an enlarged waveform, in which a soft switching technology reduces not only a switching loss and the level of conductive electromagnetic interference (EMI), so switching stress is also necessary in terms of EMI. Reference numerals ‘425’ and ‘426’ indicate a driving voltage waveform and a current waveform of an IGBT and reference numerals ‘427’ and ‘428’ indicate a primary current waveform of a transformer. In an embodiment of the present invention, a one-loop treatment coil and a two-loop treatment coil propose an effective method for treating simultaneously a lesion of herpes zoster and a lesion of PHN.

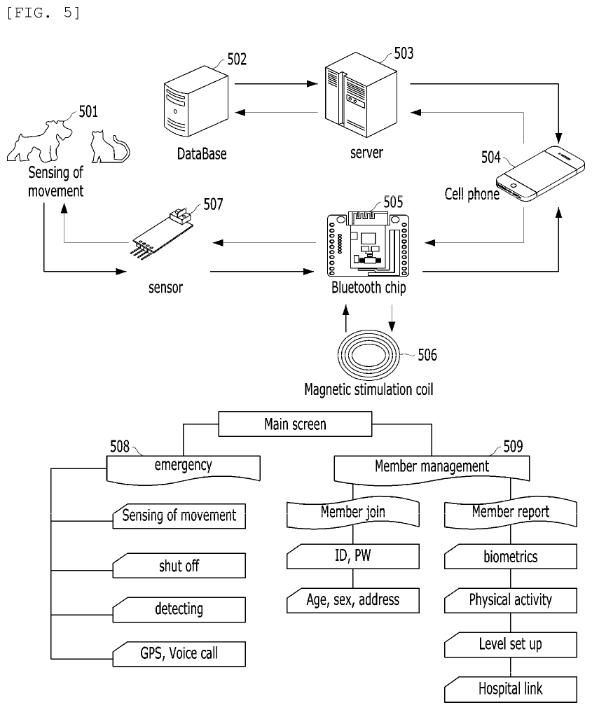

FIGS. 5 to 7 are views showing a process for treatment, communication, control, and connection with an associated person.

First, in order to handle an emergency such as herpes zoster and pain treatment using a CCD-CMOS sensor 507 and a skin and muscle conductivity sensor and manage a quick treatment performance through a cell phone, for an animal patient of reference number 501, the method reduces economic, bodily, and mental burdens and transmits database data sent and accumulated from the sensors to a database 502, thereby being able to counsel the patient, perform treatment, and receive special services through connection with facilities. Treatment is performed and locations are tracked using the sensors and the cell phone, and bio information and data are collected through a cell phone 504 and a sensor, using the database 502, and are transmitted to a medical facility through a wire/wireless system, so doctors, specialists, and guardians participate in treatment and cooperatively provide services, and uploading, feedback, disease study, and applications of a patient management system are implemented.

The apparatus proposed in an embodiment of the present invention is a system that is not limited by place and time, provides various monitoring areas, sets locations, accesses the server 503 through a wire/wireless communication network, and transmits a disease state and location information to the cell phone 504 of a guardian registered on the server 503 together with information stored in the database 502 when it goes out of the area. Bluetooth of reference numeral 505 is sensed by mounting the CCD-CMOS sensor 507 and skin and muscle conductivity sensors, it is changed into an activity suitable for an emergency, and monitoring may be added to a situation that may occur by adding GPS and voice recognition functions to the cell phone. For tracking, diagnosing, and emergency treating of a disease of a GUI (graphic user interface) type of reference numeral ‘508’, a data system is constructed when a problem is generated in a lesion; when an ID and a password is input to the server 503 from an external special organization, a member management monitor, and the cell phone 504, member information is stored in the database 502, the homepage of the external organization is accessed using Urlparse, and connection information of a guardian and a medical facility is transmitted to the primates herpes zoster and pain treatment apparatus and the cell phone 504 through the treatment coil 506.

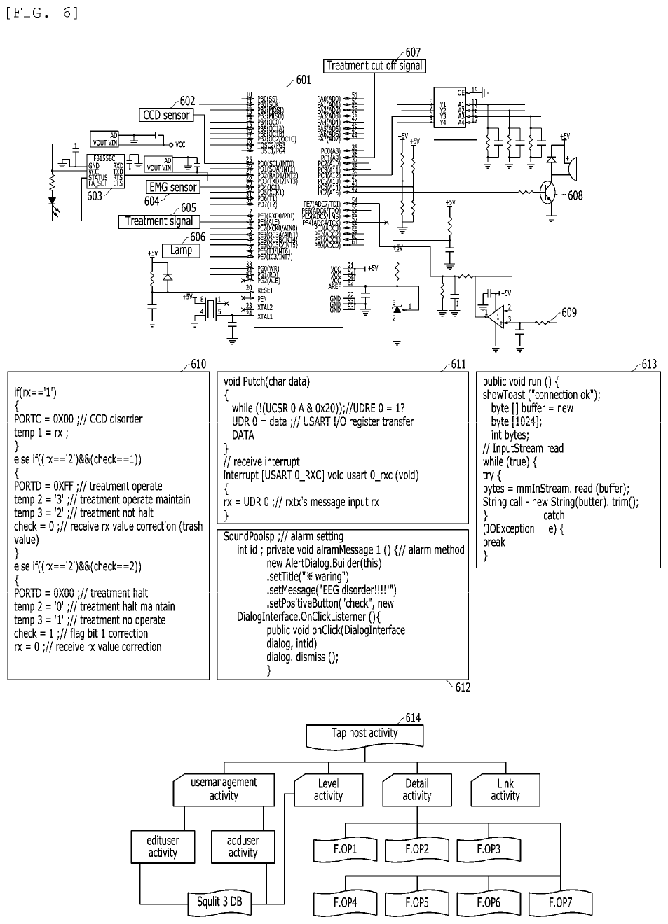

Referring to FIG. 6, according to the operation environment, a microprocessor 601 was operated by cooperation of the CCD-CMOS sensor 602, a skin, and the conductivity sensor 604, etc., MSP430 and AVR series can be used, ATmega128 was used in an embodiment of the present invention, seven ports (A-G) are provided, 8 bits excluding the G port can be used, broadcast and URL that are open sources are used in response to signal of ATmega128, a homepage is liked, and a method of making an emergency phone call can be used in an unbearable emergency using ACT CALL. According to the operation principle, when there is a problem in activity and there is no motion, LEDs are turned on, when a danger is sensed through a sensor 609 using an application of a cell phone, the danger is recognized, the LEDs are turned on, and when a warning signal of reference numeral 608, sensor, etc. are sensed by a cell phone application through Bluetooth 603, a related people are informed, whereby bidirectional communication is possible through a remote operation using a web and a phone application.

In Bluetooth, when data of sensor reach a USART I/O register, a reception interrupt is called, stored in a reception message RX, and transmitted/received to/from a cell phone, a received signal of a sensor module is input to ATmega128, an input value and a signals remotely controlled by a cell phone and a sensor are converted into digital signals and stored and analyzed in the reception RX, and then output values are output.

5V and 30˜50 mA are applied to Vcc 603, ATmega128 is connected, 7th and 8th pins are used for transmission/reception, Status is an On/Off pin, Tx of the microprocessor is connected to the RX, a received analog signal of the sensor module is converted into a digital signal, a 1-bit character is transmitted, an ADC value of the RX is read out, a light is turned on when a problem of reference number 610 is sensed, communication of an alarm code and Bluetooth is performed in 1 bit through a USART I/O register, an interrupt is used, is it stored in a message form in the RX, a code of reference numeral 611 is transmitted and received after a problem of the sensor is sensed, the sensor module call a timer from a source code stored in Atmega128, uses a Bluetooth module for a predetermined time (Test time: 5 Min), and transmits an emergency code to the cell phone, reference numeral 612 sends an emergency alarming code to the cell phone, an application informs reference numeral 613 of the problem with the sensor, and a musculoskeletal treatment, animal shingles, and pain treatment apparatus of reference numeral 605 and 607 is operated to performed treatment and then stopped. When a signal of Bluetooth is received, it is changed into activity that is an execution unit configuring a screen, an interface is implemented, a server socket is generated, Bluetooth search is executed when a connection request is sensed, whether there is connected pairing, a client that is an important part is connected, a socket is generated, transmission/reception is performed through the socket, and a listening socket is closed and a thread is forcibly ended when socket generation fails. Next, an input/output stream is set, a UUID protocol is used, a code configuring an environment that can communicate with Bluetooth is set, activity configuration is developed using Java and Xml in an android platform in application design, the OS of a cell phone uses Android, an application production method uses Eclipse and JDK, the version is 2.3.3 gingerbread, the development environment is a Jetty server and an Elasticsearch database system, and a DB uses Elastic Search and database SQLite having Android as a search engine.

According to the application structure, reference numeral 614 for five services of a CCD sensor, muscle conductor, operation sensing, GPS, and emergency phone call that are performed after a signal is transmitted to a cell phone through Bluetooth, and diagnosis and management information is configured by eight activities, there is an activity configuration in the previous page, it is implemented in a server in HostActivity, log-in and a member information storage method are implemented in UserManagement, Tomcat that is an existing server requires many settings, a simple Jetty built-in server and Java language are used, and cell phone application is not operated when an emergency application requested by a client sets a corresponding right to an Manifast.xml file in an actual operation and then does not designate the right.

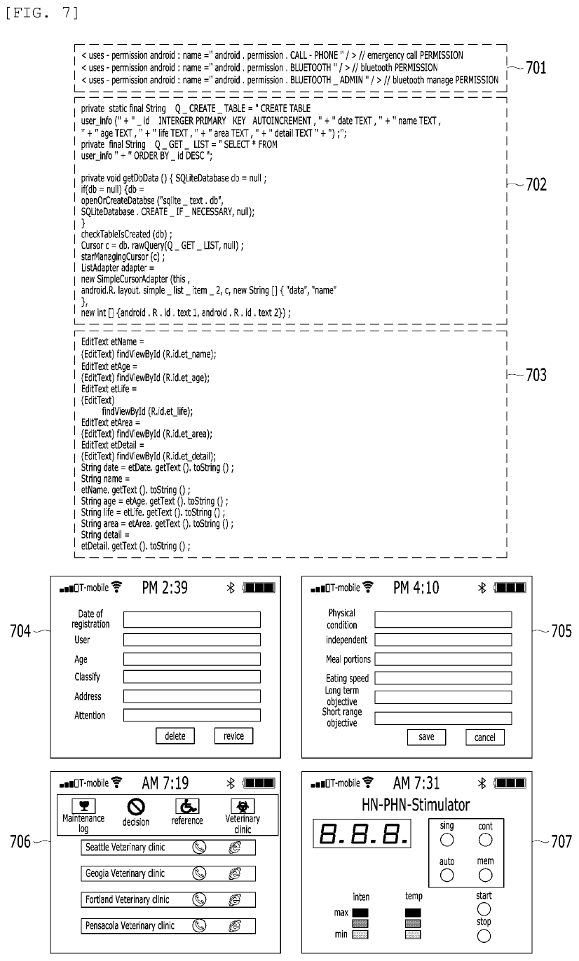

Referring to FIG. 7, for creating a database, a use log-in button and a member jointing button are made using an Oncreate method of reference numeral 701, EditText such as an ID, a password, an age, an address, a house type, daily life, and life information is made, repeatition of IID and password information is checked using a SetOnClicklistener method, a DB database of reference numeral 702 is created, a code configures a table, a table User info is created, user IDs are automatically increased, a test type key value is added, a table value and a key value are received from Sqlite_test, and a reference numeral 703 receives a list of Key values from EditText as a code in a process of receiving and updating data in a table created by receiving user input, stores it as a string value, and then stores it in a DB file. According to a result of implementing a mobile application, life and healthcare information are collected and recognized with concerned persons and guardian for early diagnosis and treatment through reference numeral 704, and can be referred to for external evaluation indexes such as neurology, BPSD, daily life activity, and performance ability, information can be supported from external instructions, disease progression is continuously monitored, and it is possible to safely cope with an emergency when an emergency occurs. Further, when a user name, a house type, a name, an age, and a dwelling area are input and stored in an image implemented by data collection and support by a diagnosis support function, a diagnosis history image of an external medical organization shows up, and weekly and monthly collected data are data to be used for diagnosis and treatment management, whereby it is possible to diagnose shingles and verify diagnosis of pain treatment after shingles.

According to the image implemented in reference numeral 705, family members or guardians can periodically input symptoms and performance abilities that are shown in daily life, and can evaluate activities, mental, meals, excretion, bathing, wearing, cleanness, etc. in levels (1˜5) on the basis of observed results, diagnosis evaluation is downloaded as an external index and referred to for level determination when observation input such as eating activity, the amount of meal, a speed, and the degree of independence is finished, and information about alternative mediation treatment such as treatment, medicinal treatment, correction support, and whether facility program support is provided is provided through short and long-term objects. Further, according to an image of a connected treatment support of reference numeral 706, when in-house treatment is difficult I accordance with a management support function and a level determination result by observation, it is possible to provide a help for smooth treatment by connecting patient information to a close animal hospital or a special clinic, and appropriate mediation and connected treatment based of determination by a behavior and BPSD doctor are proposed. Reference numeral 707 performs early treatment by setting treatment in accordance with diagnosis and situations through communication between a cell phone and an apparatus.

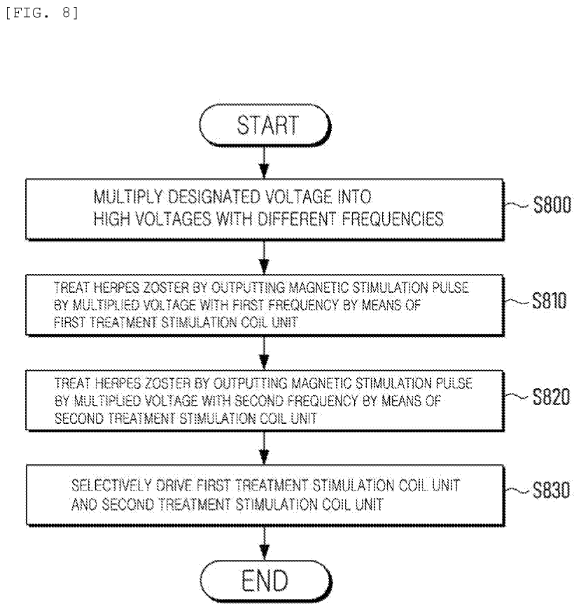

FIG. 8 is a flowchart showing a driving process of a primates herpes zoster treatment apparatus according to an embodiment of the present invention.

Referring to FIG. 8 with FIG. 1 for the convenience of description, the primates herpes zoster treatment apparatus shown in FIG. 1 according to an embodiment of the present invention multiplies a designated voltage into high voltages with different frequencies (S800). It is possible to use a transformer to generate the high voltages and a full-wave Cockcroft Walton circuit for multiplication. Multiplication voltage generation may be similar to the operation principle of a charge pump circuit.

In the primates herpes zoster treatment apparatus 90 according to an embodiment of the present invention, a stimulation coil unit is divided into two parts for herpes zoster treatment and pain treatment after herpes zoster treatment. The dual stimulation coil unit can use high voltages differently multiplied and multiplying can be controlled by a controller. Alternatively, the dual stimulation coil unit can use a one-loop coil and a two-loop coil.

Further, the first treatment stimulation coil unit of the primates herpes zoster treatment apparatus 90 treats herpes zoster by outputting a magnetic stimulation pulse by a multiplied voltage with a first frequency (S810).

On the other hand, the second treatment stimulation coil unit of the primates herpes zoster treatment apparatus 90 treats herpes zoster post-pain by outputting a magnetic stimulation pulse by a multiplied voltage with a second frequency (S820).

Further, the primates herpes zoster treatment apparatus 90 selectively drives the first treatment stimulation coil unit and the second treatment stimulation coil unit (S830). In other words, when treatment for herpes zoster is performed, it is possible to perform treatment using a magnetic stimulation pulse by the first voltage by selecting the first treatment stimulation coil unit. Further, when herpes zoster post-treatment is performed, it is possible to perform treatment using a magnetic stimulation pulse by the second voltage by selecting the second treatment stimulation coil unit.

This selection may be achieved by selecting buttons, but may be achieved by voice instructions such as voice recognition, and the controller (e.g., an MPU, a CPU) can selectively adjust the magnetic stimulation pulses by receiving control instructions from user.

Other than the above description, the primates herpes zoster treatment apparatus 90 can perform various operations, and other details were sufficiently described above and refer to the above description.

Even through all components of embodiments of the present invention are combined in one unit or operated in combination in the above description, the present invention is not limited thereto. That is, the all components may be selectively combined and operated within the scope of the present invention. Further, although all the components may be implemented as individual hardware, some or all of the components may be selectively combined as computer programs having program modules that perform some or all of functions combined in one or several items of hardware. Codes and code segments of the computer programs may be easily inferred by those skilled in the art. The computer programs may be stored in a nontransitory computer readable media and read out and executed by a computer, thereby achieving embodiments of the present invention.

The nontransitory computer readable media is not a media that stores data for a short time such as a register, a cache, and a memory, but a media that can semipermanently store data and can be read out by a device. In detail, the programs may be stored and provided in a nontransitory computer readable media such as a CD, a DVD, a hard disk, a Blu-ray disc, a USB, a memory card, and a ROM.

Although exemplary embodiments of the present disclosure were illustrated and described above, the present disclosure is not limited to the specific exemplary embodiments and may be modified in various ways by those skilled in the art without departing from the scope of the present disclosure described in claims, and the modified examples should not be construed independently from the spirit of the scope of the present disclosure.

What is claimed is:

1. A primates herpes zoster treatment apparatus comprising:
a multiplication voltage generator multiplying a designated voltage into voltages with different high frequencies;
a first treatment stimulation coil unit treating herpes zoster by outputting a magnetic stimulation pulse by the multiplied voltage with a first frequency;
a second treatment stimulation coil unit treating a herpes zoster post-pain by outputting a magnetic stimulation pulse by the multiplied voltage with a second frequency; and
a controller selectively driving the first treatment stimulation coil unit and the second treatment stimulation coil unit.

2. The primates herpes zoster treatment apparatus of claim 1, wherein the first treatment stimulation coil unit and the second treatment stimulation coil unit are operated with a switching unit that is controlled to perform switching by the controller.

3. The primates herpes zoster treatment apparatus of claim 2, wherein the multiplication voltage generator comprises a high-voltage multiplier multiplying voltage into high voltages with different frequency and then providing the voltages to the switching unit.

4. The primates herpes zoster treatment apparatus of claim 3, wherein the multiplication voltage generator comprises:
a start current compensator compensating for a start current by the designated voltage;
a transformer generating a high voltage from the designated voltage; and
a ZCS resonance inverter connected to a primary coil of the transformer and the start current compensator and performing a resonance operation by ZCS (zero-current switching).

5. The primates herpes zoster treatment apparatus of claim 4, wherein when the ZCS resonance inverter is operated, voltage induced by a secondary coil of the transformer is input to the high-voltage multiplier.

6. The primates herpes zoster treatment apparatus of claim 3, wherein the multiplication voltage generator uses a full-wave Cockcroft Walton circuit as the high-voltage multiplier.

7. A method of driving a primates herpes zoster treatment apparatus, the method comprising:
multiplying a designated voltage into voltages with different frequencies by means of a multiplication voltage generator;
treating herpes zoster by outputting a magnetic stimulation pulse by the multiplied high voltage with a first frequency by means of a first treatment stimulation coil unit;
treating a herpes zoster post-pain by outputting a magnetic stimulation pulse by the multiplied high voltage with a second frequency by means of a second treatment stimulation coil unit; and
selectively driving the first treatment stimulation coil unit and the second treatment stimulation coil unit by means of a controller.

8. The method of claim 7, further comprising operating the first treatment stimulation coil unit and the second treatment stimulation coil unit with a switching unit that is controlled to perform switching by the controller.

9. The method of claim 8, wherein the multiplication voltage generator uses a high-voltage multiplier multiplying voltage into high voltages with different frequency and then providing the voltages to the switching unit.

10. The method of claim 9, wherein the multiplying comprises:
compensating for a start current by the designated voltage by means of a start current compensator;
generating a high voltage from the designated voltage by means of a transformer; and
performing a resonance operation by ZCS by means of a ZCS resonance inverter connected to a primary coil of the transformer and the start current compensator.

11. The method of claim 10, wherein when the ZCS resonance inverter is operated, voltage induced by a secondary coil of the transformer is input to the high-voltage multiplier.

12. The method of claim 9, wherein the multiplication voltage generator uses a full-wave Cockcroft Walton circuit as the high-voltage multiplier.
